(19) United States
(12) Patent Application Publication
(10) Pub. No.: US 2020/0030764 A1
(43) Pub. Date: Jan. 30, 2020

(54) SYSTEM TO CONVERT CELLULOSIC MATERIALS INTO SUGAR AND METHOD OF USING THE SAME

(71) Applicants: Benjamin Slager, Delray Beach, FL (US); Peter James Cohen, West Palm Beach, FL (US)

(72) Inventors: Benjamin Slager, Delray Beach, FL (US); Peter James Cohen, West Palm Beach, FL (US)

(21) Appl. No.: 16/044,012

(22) Filed: Jul. 24, 2018

(51) Int. Cl. B01J 8/00; C12M 1/00; C13K 1/02; B01J 8/10

(52) U.S. Cl. CPC B01J 8/001; C12M 45/06; C13K 1/02; B01J 8/10; B01J 2219/00182; B01J 2219/00058; B01J 2219/00162; B01J 2219/00177; B01J 2219/00029

(57) ABSTRACT

A system configured to convert cellulosic materials to sugar is provided. The system has a housing a plurality of sensors coupled to the housing configured to receive input from an internal section of the housing, and convert the input to data during operation, and a control system comprising a processor, the control system configured to configured to adjust a parameter of the housing based on the received data, wherein the received data comprises an amount of oxygen within the housing, and if the oxygen in the housing is outside a predetermined range, the control system outputs a signal to an outlet to release a predetermined amount of oxygen.

### CROSS-REFERENCE TO RELATED APPLICATIONS

This application claims the benefit of U.S. Provisional Application No. 62/536,059 filed Jul. 24, 2018, entitled System and Device to Convert Cellulosic Materials into Sugar and Method of Using the Same.

### FIELD OF THE INVENTION

The present invention relates generally to automation systems, devices and methods in use to convert cellulosic materials into sugars. More particularly, the present invention relates to certain new and useful advances, devices, systems and methods to automate and mill that can be used to make monomeric sugar with the greatest yield over time, while reducing alternative product formation or monomeric sugar loss, reference being had to the drawings accompanying and forming a part of the same.

### BACKGROUND

Cellulose is an organic compound with the formula (C₆H₁₀O₅)ₙ, a polysaccharide consisting of a linear chain of several to many thousands of β(1→4) linked D-glucose units, joined by an oxygen (ether) linkage to form long molecular chains that are essentially linear. These linkages cause the cellulose to have a high crystallinity and thus a low accessibility to enzymes or acid catalysts.

Cellulose is an important structural component of the primary cell wall of green plants, many forms of algae and the oomycetes. It occurs in close association with hemicellulose and lignin, which together comprise the major components of plant fiber cells. In addition, some species of bacteria secrete it to form biofilms. Naturally formed by plants, cellulose is the most abundant organic polymer on Earth.

Cellulose, in appropriate conditions, may be decomposed to glucose by the enzyme cellulase, and, in an alternative process, it may be hydrolyzed to glucose. The degree of polymerization for cellulose may range from about 1000 for wood pulp to about 3500 for cotton fiber, giving a molecular weight from about 160,000 to about 560,000. Conversion of cellulose from crops into biofuels such as ethanol, has been developed as a fuel source process alternative to traditional sources such as refining oil and gas. Glucose sugar may also be used for fermentation.

Hydrolysis is a reaction involving the breaking of a bond in a molecule using water. The reaction mainly occurs between an ion and water molecules and often changes the pH of a solution. In chemistry, there are three main types of hydrolysis: salt hydrolysis, acid hydrolysis, and base hydrolysis. Hydrolysis of cellulose yields a mixture of simple reducing sugars, mainly glucose. These hydrolysis products can be converted to ethyl alcohol which can be used as a liquid fuel to replace petroleum, or they can be converted to methane which can be a useful source of gasoline. In addition, products of hydrolysis can also be used to manufacture various organic chemicals presently produced from petroleum. In terms of available energy, expressed as the heat of combustion of cellulose or of the glucose of alcohol theoretically obtainable therefrom, a pound of cellulose is equivalent to 0.35 lbs of gasoline (7200 BTU).

More specifically, another known method is solid acid hydrolysis in which a solid acid material is combined with a cellulose-containing material and agitated in a mill to hydrolyzing the glycosidic bonds of the cellulose material.

For example, U.S. Pat. No. 8,062,428B2 to Blair et al describes a process that when a solid acid material is combined with a cellulose-containing material and agitated, a high yield of soluble sugars can be produced. In the process, the agitation of the material, typically in a mill, provides the kinetic energy necessary to drive the hydrolysis reaction while the solid acid material has a surface acidity that aids in hydrolyzing the glycosidic bonds of the cellulose material. In addition, when the solid acid material has a sufficient existing water content, the water of the solid acid material can provide the water necessary for the hydrolysis reaction without the need for added water.

Current mills known in the art are not optimized for this process, and thus, a need exists for an improved system and method to convert cellulosic materials into sugar.

### SUMMARY OF THE INVENTION

The following summary of the invention is provided in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

To achieve the foregoing and other aspects and in accordance with the purpose of the invention, a device for the conversion of cellulose in cellulose containing materials into sugar and a system and method for using the same.

Accordingly, it is an object of this present invention to provide an efficient and economical method for the utilization of cellulosic materials.

More specifically, it is an object of the present invention to provide a new and improved device that is easy and inexpensive to construct and is smaller and more portable than anything currently available.

Another object of the present invention to provide a new and improved device that can create monomeric sugar with the greatest yield over time.

Another object of the present invention is to provide a new and improved device that also reduces alternative product formation and monomeric sugar loss.

In exemplary embodiments, a system configured to convert cellulosic materials to sugar is provided. The system comprises a housing having at least a flight disposed therein, the housing having a steam inlet and a vacuum inlet; a plurality of sensors coupled to the housing configured to receive input from an internal section of the housing, and convert the input to data during operation; and a control system comprising a processor, the control system configured to configured to adjust a parameter of the housing based on the received data; wherein the received data comprises an amount of oxygen within the housing, and if the oxygen in the housing is outside a predetermined range, the control system outputs a signal to an outlet to release a predetermined amount of oxygen.

In exemplary embodiments, a method for optimizing conversion of a cellulosic material to sugar in a mill is provided. The mill comprises a housing and plurality of sensors, the method comprises providing a flight within the housing, the flight configured to agitate a plurality of bearings in the housing; sensing an amount of oxygen within the housing, and if the oxygen in the housing is outside a predetermined range, outputting a signal to an outlet to release a predetermined amount of oxygen and adding a predetermined amount of CO2 to optimize the reaction if the amount of oxygen is under a predetermined threshold.

Other features, advantages, and aspects of the present invention will become more apparent and be more readily understood from the following detailed description, which should be read in conjunction with the accompanying drawings.

### BRIEF DESCRIPTION OF THE DRAWINGS

FIG. 1 is a perspective side view of an embodiment showing a device, namely a mill, that can be used to convert cellulose to sugar in accordance with an embodiment of the present invention;

FIG. 2 is an internal view of the mill in accordance with an embodiment of the present invention;

FIG. 3 is a block diagram of an embodiment showing a system to induce hydrolysis to cleave the glyosidic linkage of cellulose to make monomeric sugar through use of the mill in accordance with an embodiment of the present invention;

FIG. 4 is a step-wise flow chart showing a method to use a mill to convert cellulose to sugar in accordance with an embodiment of the present invention.

### DETAILED DESCRIPTION OF THE INVENTION

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described are shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be also understood to refer to functional equivalents of such structures. The present invention will now be described in detail with reference to embodiments thereof as illustrated in the accompanying drawings

As used herein, the housing may also be referred to as “mill”.

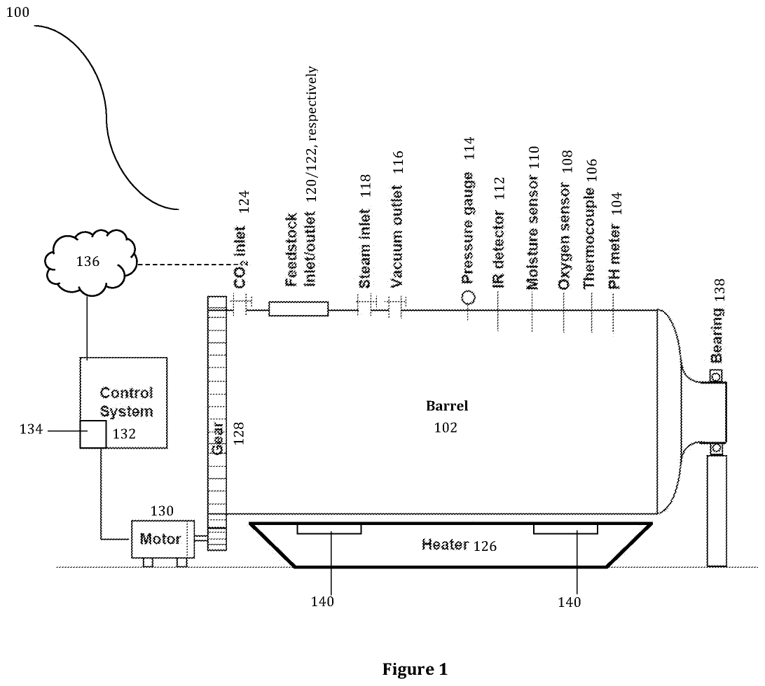

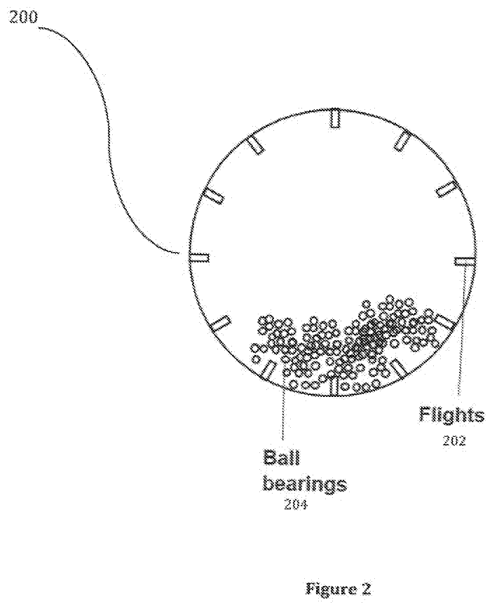

Referring now to FIG. 1, a perspective side view of an embodiment showing a device, namely a mill, that can be used in the cellulose to sugar (CTS) process in accordance with an embodiment of the present invention, is presented generally at reference numeral 100. This embodiment 100 illustrates the functional components of the mill. The mill 100 comprises a housing such as a housing 102 in which the cellulose material is placed for the purposes of utilizing the mill 100 to complete the CTS process. The interior of the housing 102 will be further described below in reference to FIG. 2.

Still referring to FIG. 1, various sensors 104-114 may be coupled to the housing 102 at optimal positions throughout the device. By “coupled” it is meant that intervening elements may be used, and the sensors may be in communication with various elements of the mill via hard wire or any known or unknown wireless technology. The sensors may comprise a pH meter 104, thermocouple 106, oxygen sensor 108, moisture sensor 110, IR detector 112 and a pressure gauge 114, each of which are coupled to a control system 132. These exemplary sensors are described herein in further detail.

The pH meter 104 is disposed inside of the housing 102, or may be disposed within a feedback testing loop. The pH meter 104 is configured to calculate the hydrogen-ion activity of the products in the mill 100 by measuring the difference in electrical potential between a pH electrode and a reference electrode. The difference in electrical potential relates to the acidity or pH of the solution. Generally speaking, pH Meters are comprised of a simple electronic amplifier and a pair of electrodes. The electrodes, or probes, are inserted into the solution to be tested. The design of the electrodes is the key part for any pH meter, and are rod-like structures usually made of glass with a bulb containing the sensor at the bottom. Once the pH meter comes in contact with the slurry an electronic amplifier detects the difference in electrical potential between the two electrodes generated in the measurement and the difference is converted to a readable scale, which are the pH units.

Because hydrolysis is catalyzed by acid sites on the catalyst, a lower pH indicates more acid sites or that sites have higher acidity, increasing the chance for hydrolysis to occur. In addition, monitoring the pH levels and assuring certain levels are met will also affect fermentation of the materials loaded into the mill 100.

In the current embodiment, the pH meter 104 is comprised of a membrane glass that has a shape and glass composition optimized to assure the best results in the current application, which in the current embodiment is comprised of a highly robust glass particularly resistant to hard chemicals and which is suitable for high temperatures. In optional embodiments, the membrane comprises high alkali glass that provides for a low alkali error for use with high temperatures and is integrated into the housing itself. In the current embodiment, the pH meter 104 will be comprised of an open junction that will allow for easy cleaning and reducing the likelihood of the sensor clogging. In optional embodiments, the pH meter 104 may be comprised of a ceramic junction. The pH meter 104 in the current embodiment has a shaft length of approximately 80-100 mm, but in optional embodiments can be a variety of sizes to account for different size housings, and has a temperate range of 0° C. to 130° C. The pH meter 104 is configured to send these data inputs to the control system 132. The pH meter 104 may also be referred to herein as a “node” for purposes of describing the automation and optimization of the processes described herein.

The thermocouple 106 is disposed inside of the housing 102, or may be disposed within a feedback testing loop. In some embodiments, the thermocouple 106 comprises two wire legs made from two different metals. The wires legs are welded together at one end, creating a junction, which is where the temperature is measured. In operation, when the junction experiences a change in temperature, a voltage is created. Overall, there are many types of thermocouples, each with its own unique characteristics in terms of temperature range, durability, vibration resistance, chemical resistance, and application compatibility. Type J, K, T, & E are commonly referred to as “Base Metal” thermocouples and are the most common types of thermocouples; on the other hand, type R, S, and B thermocouples are commonly referred to as “Noble Metal” thermocouples and are more commonly used in high temperature applications. The thermocouple is typically enclosed within a protective sheath to isolate it from the local atmosphere, which drastically reduces the effects of corrosion. The thermocouple 106 may also be referred to herein as a “node” for purposes of describing the automation and optimization of the processes described herein.

More specifically, the thermocouple 106 may be coupled to the housing 102 and used to monitor the heat temperature within the housing 102 to ensure that a high enough temperature is reached to activate the hydrolysis reaction occurring between cellulose and any agitator to make sugar; while at the same time, this temperature must also be low enough to avoid reactions that would cause the sugar to degrade. Ideally, the thermocouple 106 should be able to measure between minus 30° C. and 300° C. (−22° F. and 572° F.). The control system 132 is further coupled to various devices (e.g., heaters and coolers) that optimize the environment in which the reaction takes place such that temperature remains, for example, between 80° C. and 110° C., though other ranges may be utilized as well.

With reference still to FIG. 1, the system may further comprise an oxygen sensor 108. The oxygen sensor 108 is disposed inside of the housing 102 or may be disposed within a feedback testing loop. The oxygen sensor 108 is configured to measure the proportion of oxygen present within mill, and can measure the gas or slurry in the mill. In some embodiments, a Clark-type electrode is the most used oxygen sensor for measuring oxygen dissolved in the slurry. However, over types of sensor may be used in optional embodiments. In operation, oxygen enters the sensor through a permeable membrane by diffusion and is reduced at the cathode, creating a measurable electric current. The Clark-type sensors used herein can be made very small with a tip size of 10 μm. The oxygen consumption of such a microsensor is so small that it is practically insensitive to stirring and can be used in stagnant media such as sediments or inside plant tissue. The oxygen sensor 108 may also be referred to herein as a “node” for purposes of describing the automation and optimization of the processes described herein.

More specifically, the oxygen sensor 108 may be coupled to the housing 102 and is used to monitor oxygen levels within the housing 102. Because oxygen can cause oxidation of sugar products, the control system 132 is configured to remove oxygen from the housing 102 prior to the reaction process. To accomplish the foregoing, the oxygen sensor 108 works in conjunction with the vacuum outlet 116, which is disposed on the housing 102, such that if the oxygen sensor 108 detects a predetermined amount of oxygen (i.e. above 0% by volume or weight) within the housing 102, then the oxygen sensor 108 will communicate to the vacuum outlet 116 via the control system 132 to release oxygen out of the housing 102. In one embodiment, the vacuum outlet 116 will include vacuum pumps that will allow oxygen to be evacuated from the housing 102.

Still referring to FIG. 1, the oxygen sensor 108 also operates in conjunction with the CO2 inlet 124, which is also coupled to the housing 102 as well as the control system 132. Because CO2 is not detrimental to the reaction, inclusion of CO2 adds to water to help create carbonic acid and in turn, enhances the conversion. In optional embodiments, other gasses may also be used such as Nitrogen. Thus, if the oxygen sensor 108 detects oxygen in the housing 102 and communicates to the vacuum outlet 116 to release the same via the control system 132, the control system 132, via CO2 inlet 124 will automatically add protective CO2 gas to the housing 102 in order to maintain a positive CO2 pressure within the housing 102.

With reference still to FIG. 1, the system may further comprise a moisture sensor 110. The moisture sensor 110 is disposed inside of the housing 102, or may be disposed within a feedback testing loop. The moisture sensor used herein configured to measure the volumetric moisture in the housing 102 to ensure optimal results. Since the direct gravimetric measurement of free soil moisture requires removing, drying, and weighting of a sample, the moisture sensors measure the volumetric water content indirectly by using some other property of the soil, such as electrical resistance, dielectric constant, or interaction with neutrons, as a proxy for the moisture content. The relation between the measured property and soil moisture must be calibrated and may vary depending on environmental factors such as soil type, temperature, or electric conductivity. The moisture 110 may also be referred to herein as a “node” for purposes of describing the automation and optimization of the processes described herein.

In one embodiment of the present invention, moisture acts as a reactant to produce sugar during the CTS process and is consumed by the reaction. However, excess moisture can buffer changes in temperature as well as coat acid binding sites, thereby interfering with the acidity of the acid binding sites on the catalysts. Higher moisture levels may also deter the reaction whereas low moisture levels will reduce the chance of the reactant being consumed. As sugar is produced moisture levels in the housing 102 drop and the moisture localizes to hydrate the more hygroscopic monomeric sugars being produced. Moisture also influences the mechanical properties and rheology of the mixture. Therefore, the moisture sensor 110 ensures that the moisture levels in the housing 102 remain at a predetermined optimal level for highest output. In the present embodiment, the moisture levels may be greater than 0% but less than 50% by mass. However, this range is exemplary only and in other optional embodiments may differ. To ensure the foregoing moisture levels are maintained, a steam inlet 118 is disposed on the housing 102 and is used to disperse additional steam into the housing 102. Steam is used for even dispersal to the acid sites minimizing condensation and localization. In this way, the moisture sensor 110 may communicate via the control system 132 with the steam inlet 118 to disperse additional steam into the housing 102 when optimal.

With reference still to FIG. 1, the system may further comprise an infrared (IR) detector 112. The IR detector 112 is disposed inside of the housing 102, or may be disposed within a feedback testing loop. In embodiments, the IR detector 112 may be thermal or photonic (photodetectors). The IR detector 112 is coupled to the housing 102 and may be used to analyze the material in the housing 102 in approximately real time. In one embodiment, the infrared detector 112 may be ported into the housing 102 so that samples can be passed through a separate tubing and monitored through in the tubing as samples pass through the separate port. In some embodiment, a sapphire window may be used to show the sample passing through, the IR detectors disposed proximate the window. The IR detector 112 provides the following exemplary data, the list not being exhaustive: protein content, cellulose, starch and monomeric sugar content, lignin content, and ash and oil content. In other embodiments, algorithms may be used to automate responses through the control system 132 for all the properties mentioned herein. A fully autonomous system that optimizes conditions in the mill is disclosed herein via a control system 132. Exemplary wavelengths comprise 880 nm, 935 nm and 940 nm. The IR detector 112 may also be referred to herein as a “node” for purposes of describing the automation and optimization of the processes described herein.

The system may further comprise a pressure gauge 114. The pressure gauge 114 is disposed inside of the housing 102, or may be disposed within a feedback testing loop. The pressure gauge may comprise any sensor configured to measure the pressure of a housing 102. Exemplary sensors comprise piezoelectric, capacitate and optical. In exemplary embodiments, the pressure within the housing 102 may be preferably in a rage of approximately −10 psi to 50 psi. The pressure required to induce hydrolysis is created by the impacts of the media within the housing 102, which is also under pressure from the addition of a headspace used to reduce side reactions, such as oxidation. The control system 132 is configured to monitor and control pressure. The pressure gauge 114 may also be referred to herein as a “node” for purposes of describing the automation and optimization of the processes described herein.

The sensors listed are only exemplary in nature, as other sensors, known and unknown, may be employed. Examples include any acoustic, chemical, thermal sensors, and the like. Specific examples of chemical sensors could track reaction progress and product formation and characterization include a mass spectrometer, or a fourier transform near infrared spectrometer. All the sensors 104-114 may also be coupled to the control system 132 to communicate to the other systems and devices coupled to the housing 102, all of which are further described herein.

Still referring to FIG. 1, a feedstock inlet 120 and a feedstock outlet 122 are disposed on or coupled to the housing 102 and are used to draw feedstock into the housing 102 and to evacuate feedstock out of the housing 102, respectively. In the present embodiment, the feedstock inlet 120 and feedstock outlet 122 are operated using a vacuum system that creates positive and negative pressure in the housing 102. In other embodiments, the feedstock inlet 120 and feedstock outlet 122 may be controlled via electronic systems and coupled with the control system 132. In other optional embodiments, gravity or hopper and auger systems may be used to draw feedstock in or out.

Still referring to FIG. 1, a heater 126 is coupled to the base of the housing 102. While much of the heat required for the CTS process to occur based on the friction created within the housing 102 during the process, optionally, the housing may be heated by the heater to optimize conditions for the reaction. The heater can be a space heater, heating blanket surrounding the housing, or internal heating coil, etc. In other embodiments, specifically large commercial settings where the mill is in continuous use, heat sinks may be coupled to the housing to cool the housing 102. In even other embodiments, the cooling process may be carried out using fans coupled to the housing 102 and controlled via the control system 132. The heater 126 may also comprise cooling fans 140 or may comprise cooling elements to cool the housing 102 if it is too warm.

In an embodiment of the present invention, the control system 132 is comprises a programable logic controller (PLC) 134, which is an industrial digital computer that has been modified and adapted to be used for the control of manufacturing processes. The main difference from most other computing devices is that PLCs are intended-for and therefore tolerant-of more severe conditions (such as dust, moisture, heat, cold), while offering extensive input/output (I/O) to connect the PLC to sensors and actuators. PLC input may comprise simple digital elements such as limit switches, analog variables from process sensors (such as temperature and pressure), and more complex data such as that from positioning or machine vision systems. PLC output may comprise elements such as indicator lamps, sirens, electric motors, pneumatic or hydraulic cylinders, magnetic relays, solenoids, or analog outputs. The input/output arrangements may be built into a simple PLC, or the PLC may have external I/O modules attached to a fieldbus or computer network that plugs into the PLC. In embodiments, the PLC is configured to automate the CTS process to ensure optimized parameters of each sensed variable are in acceptable ranges to maximize output.

The automaton system may be open loop or closed loop. In closed loop control, the control action from the controller is dependent on the process output. As an example, if the temperature or IR sensor sense the temperatures is outside of an input range, the PLC 134 will send an output the heater 126 to either heat up or cool down the housing. As such, the closed loop PLC controller therefore has a feedback loop which ensures the controller exerts a control action to give a process output the same as the “reference input” or “set point.”

The system 100 further comprises a network 136 which is in communication with the PLC, each of the sensors 104-114, the heater/cooler 126, motor 130, and gear 128. The system and method of wireless data transfer from each of the nodes back to the PLC 134 may occur via wire or wireless protocol. The system 100 further comprises a plurality of computer systems which are interconnected through a network 136. The PLC 134 may have various hardware I/O devices for data acquisition. Using a real-time protocol, the PLC 134 adds one or more blocks of data to a packet and transmits the packet to every client which has subscribed to the data

Still referring to FIG. 1, the control system 132 is further coupled to a motor 130, that in turn, is coupled to a gear 128, the gear being coupled to the housing 102. The gear 128 and motor 130 are configured to turn the housing to turn the housing 102 at the optimal revolutions per minute and is able to change speeds and power outputs over time. In optional embodiments, the motor may also be configured to move the housing in all directions in any plane of movement, such that the housing can shake, roll, pitch, yaw, and the like. On an opposite end, a bearing 134 is connected to a base and is configured to allow for all ranges of motion (e.g., ball bearing, roller bearing, and the like).

In one embodiment, the control system 132 is a microcontroller that receives the data from sensors 104-114 and automatically responds to control certain predefined or predetermined parameters, which may be established and input by the operator. Real-time or approximately realtime measurements will allow for real time or approximate real time adjustments to ensure the mill 100 operates optimally. The gear 128, motor 130, and control system 132 operate together to alter the revolutions per minute as needed to adjust the torque and power of the housing 102 based upon sugar production and responses from the parameter monitoring. The gears and motor may be further configured to move the housing in other directions as well. In another example, if the thermocouple 106 sends a reading to the control system 132 that the temperature is too cold, then the control system 132 will send a corresponding signal to the heater 126 to heat the housing 102. The control system 132 is configured to allow the user to input predefined ranges for each of the above properties.

Furthermore, the control system may be configured for deep learning algorithms, such as deep learning recurrent neural network. In this way, the gates and may be self-tuning to evaluate and maximize output. This, over time, further improves and optimizes output.

Referring now to FIG. 2, an internal view of the housing 102 is illustrated in accordance with an embodiment of the present invention is presented generally at reference numeral 200. The inner wall of the housing 102 may be protected with ceramic liner to reduce wear. In this embodiment, twelve flights 202 made from steel, aluminum that are coupled to the interior of the housing 102 equidistant from each other. In the present embodiment, the flights 202 are each 5 cm in length and 0.5 cm in width to allow for both good mixing and the best distribution of impact energies, thereby allowing hydrolysis to occur. Each of the flights 202 are shaped in a rectangle which is important as is increases the number of effective impacts, minimizes the dead space and serves to strength the housing 102. In other embodiments, the flights 202 may be in a different material (e.g., carbon, metal, iron) and a different size (e.g., 4 meters long and 1 meter wide). In optional embodiments, the flights 202 may be of other shapes that still promote product formation, for example triangles of varying degrees. In other optional embodiments, there may also be a different amount of flights 202. The optimal number of flights 202, as well as their size, shape and composition material may vary depending on the size of the housing 102 to ensure optimal sugar production and product longevity. In even other embodiments, agitators may also be including in addition to the flights 202 to promote optimal mixing and to increase the number of impacts that cause hydrolysis. Furthermore, the flights may be spaced differently depending upon the type of use. As an example, they may be trapezoidal in shape as opposed to square or rectangle. A mix of shares may be used as well.

Still referring to FIG. 2, agitators 204 may comprise the ball bearings that are housed within the housing 202 and are left in a loose manner such that they are free to move around the interior of the housing 102 as it rotates. The agitators 202 are used to create the pressure that is necessary for the reaction to occur. In the current embodiment, the agitators are ball bearings are made from metal. In other embodiments, they may be made from aluminum or materials such as polymer and polymer composites. In other embodiments, they may be made from ceramic materials and they may be of any useful shape and size.

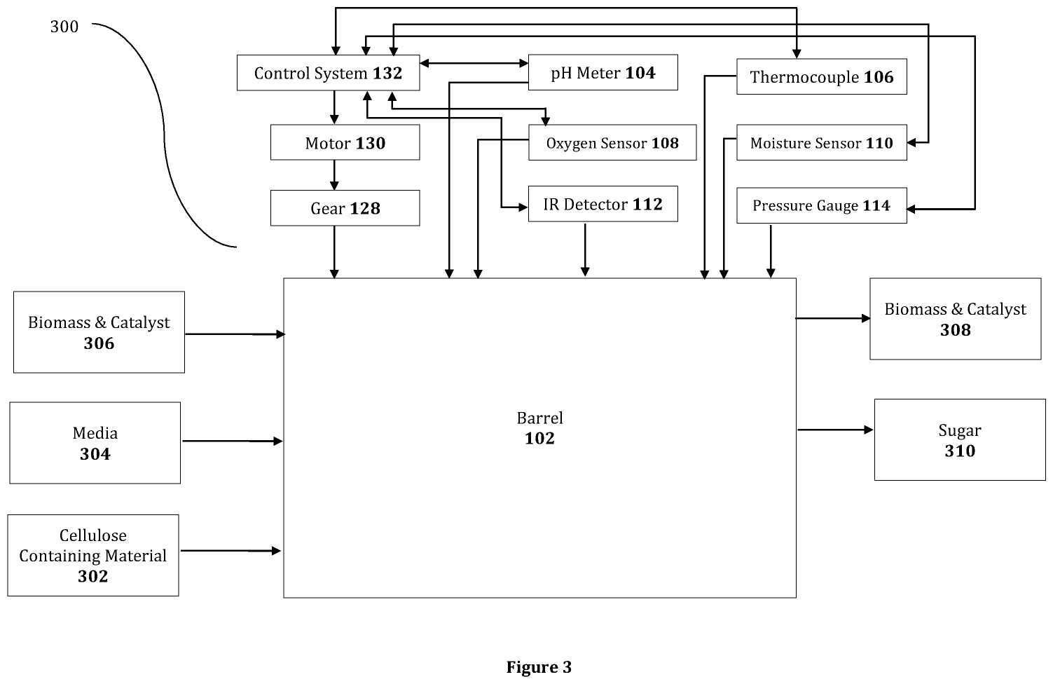

Referring now to FIG. 3, a block diagram illustrating a system to induce hydrolysis to cleave the glyosidic linkage of cellulose to make monomeric sugar through use of the mill in the CTS process in accordance with embodiments of the present invention is shown generally at 300. This figure will be used to describe a method herein, as well. The method begins with the addition of the cellulose containing material 302, media 304 and biomass and catalyst 306 into the housing 102. As described above, the cellulose containing material 302 generally includes the cell wall of green plants, lignin, many forms of algae and the oomycetes. Cellulose containing material 302 may also be obtained from the bark, wood or leaves of plants in addition to plant based material. Here, media 304 is used to create the pressure required by the reaction. There are optimal media choices based on longevity and effectiveness. The size of the media 304 is an important consideration allowing more or less space for bulk feedstock and catalyst. The size of the media 304 will also affect the amount of surface area for the reaction to occur. The media 304 chosen will also have an optimal material with physical properties including density and hardness that will be optimized for the conversion process. There is also an optimal ratio of media 304 to volume and aspect ratio of the housing 102. There is also an optimal ratio of media 102 to feedstock for monomeric sugar production. The media 304 is also required for optimal sugar production.

Still referring to FIG. 3, the biomass and catalyst 306 are introduced to the housing 102 through a port. The control system 132 is configured to read the sensors 104-114 coupled to the housing 102 to make any adjustments to the amount of biomass and catalyst 306 utilized to ensure for optimal sugar production as the biomass and catalyst 306 added to the housing 102 at amounts and ratios that result in the highest rate of hydrolysis. At the conclusion of the CTS process, the biomass and catalyst 308 are separated from the sugar 310 and both exit from the housing 102 at different exit ports.

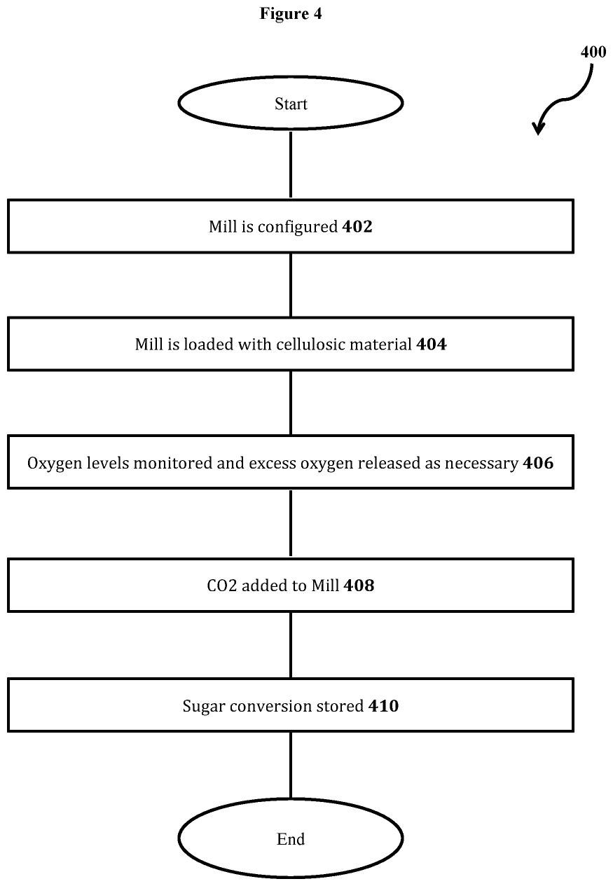

With reference now to FIG. 4, a method for using a mill to convert cellulose to sugar in accordance with one embodiment of the present invention, is presented generally at 400. The mill discussed in this method will have a plurality of flights disposed therein. In optional embodiments, the mill may have a different internal structure. The method begins with the mill being configured to shake, roll, or both, to agitate a plurality of bearings within the mill 402. Once configured, the mill is loaded with cellulosic material and the process begins 404. During the conversion process, the sensors coupled to the mill are used to monitor the oxygen levels within the housing, and if the sensors determine that the oxygen in the housing is outside a predetermined range, then the system will open the outlet to release a predetermined amount of oxygen 406. Once oxygen levels are calculated and adjusted as necessary, predetermined amounts of CO2 are added to the mill to optimize the reaction if the oxygen levels are found to be less than a predetermined threshold 408. Once the oxygen is released and the CO2 added, as necessary, then the mill will finish the conversion and the sugar is stored 410.

Specific configurations and arrangements of the invention, discussed above with reference to the accompanying drawing, are for illustrative purposes only. Other configurations and arrangements that are within the purview of a skilled artisan can be made, used, or sold without departing from the spirit and scope of the invention. For example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures.

While the present invention has been described in connection with what are presently considered to be the most practical and preferred embodiments, it is to be understood that the present invention is not limited to these herein disclosed embodiments. Rather, the present invention is intended to have various modifications and equivalent arrangements included within the spirit and scope of the appended claims.

Although specific features of various embodiments of the invention may be shown in some drawings and not in others, this is for convenience only. In accordance with the principles of the invention, the feature(s) of one drawing may be combined with any or all of the features in any of the other drawings. The words “including,” “comprising,” “having,” and “with” as used herein are to be interpreted broadly and comprehensively, and are not limited to any physical interconnection. Moreover, any embodiments disclosed herein are not to be interpreted as the only possible embodiments. Rather, modifications and other embodiments are intended to be included within the scope of the appended claims.

We claim:

1. A device configured to convert cellulosic materials to sugar, the device comprising:
a housing having at least a flight disposed therein, the housing having a steam inlet and a vacuum inlet;
a plurality of sensors coupled to the housing configured to receive input from an internal section of the housing, and convert the input to data during operation; and
a control system comprising a processor, the control system configured to configured to adjust a parameter of the housing based on the received data;
wherein the received data comprises an amount of oxygen within the housing, and if the oxygen in the housing is outside a predetermined range, the control system outputs a signal to an outlet to release a predetermined amount of oxygen.

2. The system of claim 1, wherein the plurality of sensors comprises at least one pressure sensor.

3. The system of claim 1, wherein the plurality of sensors comprises at least one temperature sensor, wherein the at least one temperature sensor is a thermocouple and is further configured to output a signal to the control system if the pH is outside of a predetermined range.

4. The system of claim 1, wherein the plurality of sensors comprises a pH meter configured to calculate the hydrogen-ion activity of the products in the housing, and further configured to output a signal to the control system if the temperature is outside of a predetermined range.

5. The system of claim 1, wherein the plurality of sensors comprises an oxygen sensor configured to calculate the oxygen content in the housing, and further configured to output a signal to the control system if the oxygen is outside of a predetermined range.

6. The system of claim 5, further comprising a carbon dioxide inlet coupled to the housing and a carboned dioxide source, the carbon dioxide being added to create carbonic acid and in turn, enhances a conversion of cellulose to sugar.

7. The system of claim 5, wherein if the if the oxygen sensor detects oxygen in the housing and communicates to the outlet to release the same via the control system, the control system is configured to, via CO2 inlet, automatically add protective inert CO2 gas to the housing.

8. The system of claim 1, wherein the plurality of sensors comprises a moisture sensor configured to calculate the moisture of the products in the housing, and further configured to output a signal to the control system if the moisture is outside of a predetermined range.

9. The system of claim 8, wherein as moisture is consumed to produce the sugar, the moisture sensor and control system are configured to ensure that the moisture level remain at a predetermined optimal level for sugar output using a steam inlet to disperse steam into the housing.

10. The system of claim 1, wherein the plurality of sensors comprises an infrared detector configured to monitor a content of a material within the housing.

11. The system of claim 1, further comprising:
a feedstock inlet disposed on the housing;
and a feedstock outlet disposed on the housing;
wherein each of the inlet and outlet are configured to draw cellulosic material into the housing and to evacuate a sugar out of the housing, respectively using a vacuum system that creates positive and negative pressure in the housing.

12. The system of claim 1, further comprising a heating element, cooling element, or both, positioned proximate the housing.

13. The system of claim 1, further comprising a gear and a motor coupled to the housing and configured to provide a motive force to rotate the housing.

14. The system of claim 1, further comprising a bearing coupled to a shaft, the shaft being attached to the housing.

15. A method for to optimizing conversion of a cellulosic material to sugar in a mill, the mill comprising housing and plurality of sensors, the method comprising:
providing a flight within the flight, the flight configured to agitate a plurality of bearings in the housing;
sensing an amount of oxygen within the housing, and if the oxygen in the housing is outside a predetermined range, outputting a signal to an outlet to release a predetermined amount of oxygen;
adding a predetermined amount of CO2 to optimize the reaction if the amount of oxygen is under a predetermined threshold to optimize the conversion
